(19) United States
(12) Patent Application Publication
(10) Pub. No.: US 2006/0263827 A1
(43) Pub. Date: Nov. 23, 2006

(54) Novel endothelial cells, antibodies directed against said cells and use thereof, in particular for screening angiogenesis inhibiting substances

(76) Inventors: Jean Pierre Plouet, Toulouse (FR); Sandrine Florence Pedron, Ramonville (FR); Maitre Boube, Tourefeuille (FR)

Correspondence Address: YOUNG & THOMPSON, 745 SOUTH 23RD STREET, 2ND FLOOR, ARLINGTON, VA, 22202, US

(21) Appl. No.: 10/530,893

(22) PCT Filed: Oct. 10, 2003
(86) PCT No.: PCT/FR03/02996

§ 371 Date: Feb. 27, 2006

(30) Foreign Application Priority Data
Oct. 10, 2002 (FR) 02/12606

(51) Int. Cl. G01N 33/567; A61K 38/18

(52) U.S. Cl. 435/7.2; 514/12

(57) ABSTRACT

The invention concerns the use of a binary set comprising: endothelial cells of non-angiogenic phenotype and endothelial cells of angiogenic phenotype, for screening substances angiogenic towards endothelial cells of non-angiogenic phenotype, not substantially affecting substantially the of angiogenic phenotype endothelial cells of angiogenic phenotype, or substances angiogenic towards endothelial cells of angiogenic phenotype, not affecting substantially the endothelial cells of non-angiogenic phenotype.

The present invention relates to novel endothelial cells, as well as antibodies directed against these cells. The present invention also relates to the use of these cells, in particular for the screening of substances inhibiting angiogenesis.

Endothelial cells line the inside of all the blood vessels of an organism. These cells have a quiescent phenotype, they scarcely proliferate at all (only 0.01% of endothelial cells are engaged in cell division at any given moment). The physiological role of the endothelial cells is immense, they participate in numerous phenomena vital to the organism: blood coagulation, maintenance of arterial pressure, recruitment of circulating cells, synthesis of growth factors. When an area requires an increased supply of oxygen and nutrients it reacts by releasing soluble factors which convert the phenotype of the endothelial cells to an activated phenotype, and mitogenic factors for endothelial cells with an activated phenotype. These endothelial cells finally acquire an angiogenic phenotype and become capable of carrying out the angiogenesis program, i.e. the development of new blood vessels from the pre-existing network. This phenomenon is normal and controlled when there is an injury or in the maturation of the corpus luteum of the ovary; on the other hand, it becomes dangerous in pathological situations where angiogenesis is no longer controlled, for example in diabetic retinopathy, polyarthritis and tumor development.

It is necessary that a therapeutic agent attacks only endothelial cells participating in angiogenesis, i.e. cells with an angiogenic phenotype (Brooks et al., 1994), hence the importance of studying the angiogenic phenotype for the development of new tools in order to block angiogenesis. Up to now few studies have been carried out on cells with an angiogenic phenotype, a major difficulty being the impossibility of extracting cells with this phenotype and keeping them differentiated in culture in vitro. Endothelial cells are cultured in the presence of FGF2 in a standard manner and therefore, like any cell cultured in vitro, they lose their quiescent phenotype and acquire an activated phenotype when they proliferate in a culture dish. On the other hand, the maintenance of the angiogenic phenotype could be ensured by the addition of FGF2 to the culture medium (Boudreau et al., 1997; Battegay et al., 1994). However, the role of FGF2 in pathology is controversial.

At present, there are no models available for the study of endothelial cells which, although proliferating, either mimic the non-activated phenotype (non-angiogenic phenotype), or mimic the angiogenic phenotype.

The purpose of the present invention is to provide endothelial cells exclusively dependent on VEGF.

The purpose of the present invention is to provide a process for preparing said cells cultured on a layer of gelatin or collagen and incubated or not incubated in a culture medium the hormone and growth factor content of which is controlled.

The present invention relates to the use of a binary assembly comprising:
endothelial cells with a non-angiogenic phenotype and
endothelial cells with an angiogenic phenotype,

for the screening

of angiogenic substances vis-à-vis endothelial cells with a non-angiogenic phenotype, not substantially affecting endothelial cells with an angiogenic phenotype, or

of anti-angiogenic substances vis-à-vis endothelial cells with an angiogenic phenotype, not substantially affecting endothelial cells with a non-angiogenic phenotype.

The present invention relates to the use of a binary assembly comprising:
endothelial cells with a non-angiogenic phenotype and
endothelial cells with an angiogenic phenotype,

for the screening of anti-angiogenic substances, vis-à-vis endothelial cells with an angiogenic phenotype, not substantially affecting endothelial cells with a non-angiogenic phenotype.

The constituents of the “binary assembly” designate each and every one of the phenotypes, which in culture form tubes in response to FGF2, but only one of the phenotypes of which responds to VEGF.

The expression “endothelial cells with a non-angiogenic phenotype” designates cells incapable of organizing themselves into tubes in culture in a collagen gel in the presence of VEGF.

The expression “endothelial cells with an angiogenic phenotype” designates cells capable of organizing themselves into tubes in culture in a collagen gel in the presence of VEGF.

The expression “anti-angiogenic substances” designates any natural or synthetic substance which, added to the culture medium, inhibits the cell activity of one or both elements of the binary system described above, during at least one of the 3 following tests (described hereafter in the examples): proliferation, migration, in vitro angiogenesis, or inhibits tumorigenesis (described in the examples).

The expression “angiogenic substances” designates any natural or synthetic substance which, added to the culture medium, stimulates the cell activity of one or both of the elements of the binary system described above during at least one of the 3 following tests (described in the examples): proliferation, migration, in vitro angiogenesis, or stimulates tumorigenesis (described in the examples).

The expression “substances not substantially affecting endothelial cells with a non-angiogenic phenotype” designates any natural or synthetic substance which, added to the culture medium, does not modify the activity of the cells with a non-angiogenic phenotype during at least one of the 3 following tests (described in the examples): proliferation, migration, in vitro angiogenesis.

The expression “substances not substantially affecting endothelial cells with an angiogenic phenotype” designates any natural or synthetic substance which, added to the culture medium, does not modify the activity of the cells with an angiogenic phenotype during at least one of the 3 following tests (described in the examples): proliferation, migration, in vitro angiogenesis.

The present invention also relates to a process for screening substances capable of inhibiting the angiogenesis of endothelial cells with an angiogenic phenotype, but not substantially affecting endothelial cells with a non-angiogenic phenotype, comprising the following stages:
culture, on the one hand, of endothelial cells with a non-angiogenic phenotype and, on the other hand, of endothelial cells with an angiogenic phenotype, in order to respectively obtain a culture of endothelial cells with an angiogenic phenotype and a culture of endothelial cells with a non-angiogenic phenotype,
addition to each of the cultures defined above, of a mitogenic factor, in particular chosen from the family of the factors FGF, PDGF, VEGF or EGF, and being in particular VEGF, and of the substance to be tested, capable of inhibiting angiogenesis, and the maintenance of the above-mentioned cells in culture for a time sufficient for at least one cell division cycle to occur,
comparison of the inhibition, by the substance to be tested, of the mitogenic action of the mitogenic factor, on the one hand on the endothelial cells with an angiogenic phenotype, and on the other hand, on the endothelial cells with a non-angiogenic phenotype, making it possible to select from the above-mentioned substances to be tested what inhibits the proliferation of endothelial cells with an angiogenic phenotype, but does not inhibit the proliferation of endothelial cells with a non-angiogenic phenotype.

The present invention therefore also relates to a process for screening anti-angiogenic substances vis-à-vis endothelial cells with an angiogenic phenotype.

The expression “mitogenic factor” designates a substance capable of triggering cell proliferation.

The expression “maintenance of the cells in culture for a time sufficient for at least one cell division cycle to occur” corresponds to a minimum duration of 24 to 48 hours, according to the cell phenotype.

The expression “mitogenic action” designates the action of a mitogenic factor, namely cell proliferation.

The expression “inhibition of the mitogenic action of the mitogenic factor” designates the action of a substance inhibiting the activity of a mitogenic factor, namely cell proliferation.

Comparison of the inhibition by the substance to be tested, of the mitogenic action of the mitogenic factor, on the one hand on endothelial cells with an angiogenic phenotype, and on the other hand, on endothelial cells with a non-angiogenic phenotype is carried out according to the test of proliferation induced by the mitogenic factor on cells of each phenotype.

The present invention also relates to a binary assembly comprising:
endothelial cells with a non-angiogenic phenotype and
endothelial cells with an angiogenic phenotype,

characterized in that the endothelial cells with a non-angiogenic phenotype have at least one of the following properties:
they remain confluent when they are brought into the presence of the growth factor VEGF, without forming tubules,
they do not proliferate under the action of VEGF,
they are not protected from apoptosis by VEGF,

and in that the endothelial cells with an angiogenic phenotype have at least one of the following properties:
they form tubes when they are brought into the presence of the growth factor VEGF,
they proliferate under the action of VEGF,
they are protected from apoptosis by VEGF.

According to an advantageous embodiment of the invention, the endothelial cells with a non-angiogenic phenotype resist anti-angiogenic treatment inhibiting the phosphorylation of VEGF-R2 (see FIG. 4).

According to an advantageous embodiment of the invention, the endothelial cells with an angiogenic phenotype are sensitive to anti-angiogenic treatment inhibiting the phosphorylation of VEGF-R2 (see FIG. 4).

The term “confluent” designates the state of cells lining the surface of the support of the culture dish without leaving any empty space, or joined cells without continuity solution.

The term “growth factor” designates a substance inducing cell proliferation or differentiation. The term “tubules” designates a cell elongation followed by the organization into 2 parallel rows of cells delimiting a space in the form of a hollow tube.

The expression “not protected from apoptosis” means that cells deprived of serum die over the course of a cascade of events which is expressed by the fragmentation of the nuclei. This phenomenon is demonstrated by counting fragmented cell nuclei according to the technique described by Gavrieli (Gavrieli et al., 1992).

Apoptosis can be induced for example by deprivation of serum, or by any other means known to a person skilled in the art.

The term “angiogenic treatment” designates bringing the cells into the presence of a substance triggering an angiogenic effect, in particular VEGF.

It is verified that endothelial cells with a non-angiogenic phenotype remain confluent when they are brought into the presence of the growth factor VEGF, without forming tubules, by the in vitro angiogenesis test.

It is verified that endothelial cells with a non-angiogenic phenotype resist angiogenic treatment, by the proliferation test.

It is verified that endothelial cells with a non-angiogenic phenotype are not protected from apoptosis by VEGF, by Gavrieli's test.

It is verified that endothelial cells with an angiogenic phenotype form tubes when they are brought into the presence of the growth factor VEGF, by the in vitro angiogenesis test.

It is verified that the endothelial cells with an angiogenic phenotype are sensitive to angiogenic treatment, by the proliferation test.

It is verified that the endothelial cells with an angiogenic phenotype are protected from apoptosis by VEGF, by Gavrieli's test.

According to an advantageous embodiment, the present invention relates to endothelial cells with a non-angiogenic phenotype as defined above.

The present invention relates to endothelial cells with an angiogenic phenotype as defined above.

The present invention relates to endothelial cells as defined above, characterized in that these are endothelial cells of vessels, in particular endothelial cells of the aorta, adrenal cortex, skin, cerebrum, retina, veins or umbilical cord artery.

The present invention also relates to a process for preparing endothelial cells with a non-angiogenic phenotype as defined above, characterized in that it comprises the following stages:
incubation of endothelial cells, in particular removed from an aorta, in a medium containing neither oestradiol, nor growth factor, in particular not containing VEGF, in order to obtain clones of endothelial cells with a non-angiogenic phenotype,
removal of a clone using a micropipette, from the above-mentioned clones of endothelial cells with a non-angiogenic phenotype, and the culture of this clone until cell confluence is obtained, checked by examination using a phase contrast microscope.
selection of endothelial cells with a non-angiogenic phenotype, by verification of the phenotype of the cells obtained in the preceding stage, using the proliferation and/or migration and/or in vitro angiogenesis test.

A medium containing neither oestradiol, nor growth factor is used in order not to stimulate VEGF synthesis (Concina et al., 2000).

The medium described in the examples is advantageously used.

After the incubation stage, all the clones are removed, tested by in vitro angiogenesis and those which are “non-angiogenic”, i.e. those which do not form tubes under the action of VEGF, are selected.

The confluence of the cells is verified by microscope observation.

The proliferation test and the migration test are described in the following reference: Jonca et al. (1997).

The angiogenesis test is described in the examples.

The three tests can be used in order to characterize the clones, but the in vitro angiogenesis test can be sufficient for characterization.

The present invention also relates to a process for preparing endothelial cells with an angiogenic phenotype as defined above, characterized in that it comprises the following stages:
incubation of endothelial cells, in particular removed from an aorta in a medium containing oestradiol and a growth factor, in particular VEGF, in order to obtain clones of endothelial cells with an angiogenic phenotype,
removal of a clone using a micropipette, from the above-mentioned clones of endothelial cells with an angiogenic phenotype, and the culture of this clone until cell confluence is obtained,
selection of the endothelial cells with an angiogenic phenotype, by verification of the phenotype of the cells obtained in the preceding stage, using proliferation, migration or in vitro angiogenesis tests.

The expression “clones of endothelial cells with an angiogenic phenotype” designates a collection of cells corresponding to the required criteria.

After the incubation stage, all the clones are removed, tested by in vitro angiogenesis and those which are “angiogenic”, i.e. those which form tubes under the action of VEGF, are selected.

The present invention also relates to a polyclonal or monoclonal antibody directed against endothelial cells with an angiogenic phenotype as defined above, in particular a monoclonal antibody capable of activating or inhibiting angiogenesis.

By “antibody capable of activating angiogenesis”, is meant an antibody with angiogenic activity, as defined above. By “antibody capable of inhibiting angiogenesis” is meant an antibody endowed with anti-angiogenic activity as defined above.

The present invention also relates to a polyclonal or monoclonal antibody directed against endothelial cells with a non-angiogenic phenotype as defined above, in particular a monoclonal antibody capable of activating or inhibiting angiogenesis.

According to an advantageous embodiment of the present invention, a monoclonal antibody directed against endothelial cells with an angiogenic phenotype of the invention has the following characteristics:
it binds to the surface of endothelial cells with an angiogenic phenotype, and
it recognizes a unit present exclusively on endothelial cells with an angiogenic phenotype, in particular a membrane receptor.

It is verified that the monoclonal antibody directed against endothelial cells with an angiogenic phenotype of the invention binds to the surface of the endothelial cells with an angiogenic phenotype, by ELISA test on cells or cytometry, as defined in the examples.

It is verified that the monoclonal antibody directed against endothelial cells with an angiogenic phenotype of the invention recognizes a unit present exclusively on the endothelial cells with an angiogenic phenotype, in particular a membrane receptor, by the same tests as those indicated above.

The present invention also relates to a process for preparing a monoclonal antibody directed against angiogenic cells as defined above, capable of activating angiogenesis, characterized in that it comprises the following stages:
immunization of an animal by injection of cells with an angiogenic phenotype,
fusion between myelomas of an animal and splenocytes of an animal in order to obtain hybridomas,
culture of the hybridomas thus obtained,
cloning of hybridomas, chosen from those obtained in the preceding stage and secreting antibodies against cells with an angiogenic phenotype,
verification of the angiogenesis-activation properties of the abovementioned antibodies vis-à-vis angiogenic cells, in particular using the proliferation and/or migration and/or in vitro angiogenesis test.

The animal used for the immunization stage is in particular a mouse or a rat.

The myelomas used for the fusion originate in particular from a mouse or a rat.

The splenocytes used for the fusion originate from an animal of the same species as that from which the myelomas originate, namely in particular a mouse or a rat.

Hybridomas which secrete antibodies against the cells with an angiogenic phenotype are chosen by ELISA, or by cytometry, and the angiogenic function is demonstrated by a proliferation and in vitro angiogenesis test.

The present invention also relates to a process for preparing a monoclonal antibody directed against angiogenic cells as defined above, capable of inhibiting angiogenesis, characterized in that it comprises the following stages:
immunization of an animal by injection of cells with an angiogenic phenotype,
fusion between myelomas of an animal and splenocytes of an animal in order to obtain hybridomas,
culture of the hybridomas thus obtained,
cloning of hybridomas, chosen from those obtained in the preceding stage and secreting antibodies against cells with an angiogenic phenotype,
verification of the angiogenesis-inhibiting properties of the above-mentioned antibodies vis-à-vis angiogenic cells, in particular using the proliferation and/or migration and/or in vitro angiogenesis test.

The animal used for the immunization stage is in particular a mouse or a rat.

The myelomas used for the fusion originate in particular from a mouse or a rat.

The splenocytes used for the fusion originate from an animal of the same species as that from which the myelomas originate, namely in particular a mouse or a rat.

Hybridomas which secrete the antibodies against cells with an angiogenic phenotype are chosen by ELISA, or by cytometry, and the anti-angiogenic function is demonstrated by a proliferation and in vitro angiogenesis test.

The present invention also relates to anti-idiotypic antibodies directed against monoclonal antibodies themselves directed against endothelial cells with a non-angiogenic phenotype as defined above, or against monoclonal antibodies themselves directed against endothelial cells with an angiogenic phenotype as defined above, characterized in that they are capable of activating or inhibiting the functions performed (activation or inhibition of angiogenesis) by the antibodies as defined above.

The present invention also relates to anti-idiotypic antibodies directed against monoclonal antibodies themselves directed against endothelial cells with an angiogenic phenotype, characterized in that they are capable of activating the functions performed (activation or inhibition of angiogenesis) by the antibodies as defined above.

These antibodies therefore mimic the structures of the receptors and can be considered as circulating receptors, therefore functioning as anti-ligand antibodies. The advantage lies in the construction of anti-ligand antibodies without the nature of the ligand being known.

The present invention also relates to anti-idiotypic antibodies directed against monoclonal antibodies themselves directed against endothelial cells with an angiogenic phenotype, characterized in that they are capable of inhibiting functions performed (activation or inhibition of the angiogenesis) by the antibodies as defined above.

These antibodies therefore mimic the structures of the receptors and can be considered as circulating receptors, therefore functioning as anti-ligand antibodies. The advantage lies in the construction of anti-ligand antibodies without the nature of the ligand being known.

The present invention also relates to Fab fragments of the monoclonal or polyclonal antibodies, as defined above, and anti-idiotypic antibodies as defined above, said fragments being capable of activating or inhibiting angiogenesis.

The Fab fragments can be obtained by cleavage of the heavy chain of the antibodies, in particular by papain. They therefore bind to the receptors in the same fashion but do not dimerize them, therefore they only perform a receptor-blocking activity.

The present invention also relates to a complex between:
an antibody, as defined above, or a Fab fragment, an angiogenesis activator, as defined above, and
a radioactive element such as iodine 125 or 131, indium, yttrium or any other compound containing an ionizing particle.

Binding to the target cell, this complex can destroy it by means of the cytolytic element which is linked to it.

The present invention also relates to a complex between an antibody, as defined above, an angiogenesis activator, and a cytolytic compound, in particular a toxin.

Binding to the target cell, this complex is then internalized inside the cell and can destroy it by means of the compound which is linked to it.

The present invention relates to a process for preparing the anti-idiotypic antibodies as defined above, directed against monoclonal antibodies themselves directed against endothelial cells with an angiogenic phenotype, said process being characterized in that it comprises the following stages:
immunization of an animal by injection of monoclonal antibodies as defined above, namely angiogenic anti-cell antibodies,
fusion between myelomas of an animal and splenocytes of an animal, in order to obtain hybridomas,
culture of the hybridomas thus obtained,
cloning of hybridomas, chosen from those obtained in the preceding stage and secreting antibodies directed against the abovementioned monoclonal antibodies, used for immunization, said monoclonal antibodies being directed against the cells with an angiogenic phenotype, and
verification of the inhibition properties of the abovementioned antibodies vis-à-vis the function of activation or inhibition of the angiogenesis of the antibodies as defined above, in particular using the proliferation and/or migration and/or in vitro angiogenesis test.

The animal used for the immunization stage is in particular a mouse or a rat.

The myelomas used for the fusion originate in particular from a mouse or a rat.

The splenocytes used for the fusion originate from an animal of the same species as that from which the myelomas originate, namely in particular from a mouse or a rat.

Hybridomas which secrete antibodies directed against the monoclonal antibodies used for immunization are chosen by ELISA using the Fab fragments of the antibodies having served as immunogen as reaction substrate.

The present invention also relates to anti-anti-idiotypic antibodies directed against endothelial cells with an angiogenic phenotype as defined above, characterized in that they are capable of activating or inhibiting angiogenesis.

The present invention also relates to anti-anti-idiotypic antibodies directed against endothelial cells with an angiogenic phenotype as defined above, characterized in that they are capable of activating or inhibiting angiogenesis. Since these antibodies are directed against an antibody which mimics a receptor, they behave like mimics of the ligand. The advantage is therefore obtaining mimics of the ligand, even whilst not knowing its structure.

The present invention also relates to a process for preparing the anti-anti-idiotypic antibodies as defined above, directed against endothelial cells with an angiogenic phenotype, said process being characterized in that it comprises the following stages:
immunization of an animal by injection of anti-idiotypic antibodies as defined above, namely angiogenic anti-cell anti-antibody antibodies,
fusion between myelomas of an animal and splenocytes of an animal, in order to obtain hybridomas,
culture of the hybridomas thus obtained,
cloning of hybridomas, chosen from those obtained in the preceding stage and secreting anti-anti-idiotypic antibodies directed against cells with an angiogenic phenotype, and
verification of the properties of inhibition or activation of the angiogenesis of the abovementioned anti-anti-idiotypic antibodies, in particular using the proliferation and/or migration and/or in vitro angiogenesis test.

The animal used for the immunization stage is in particular a mouse or a rat.

The myelomas used for the fusion originate in particular from a mouse or a rat.

The splenocytes used for the fusion originate from an animal of the same species as that from which the myelomas originate, namely in particular a mouse or a rat.

Hybridomas which secrete anti-anti-idiotypic antibodies directed against cells with an angiogenic phenotype are chosen by measuring their binding to the Fab fragments of the anti-idiotypic antibodies.

The present invention also relates to a pharmaceutical composition, characterized in that it contains, as active ingredient, an angiogenesis inhibitor, chosen from an antibody as defined above, an anti-idiotypic antibody as defined above, a Fab fragment as defined above, or a complex as defined above, in combination with a pharmaceutically acceptable vector, said pharmaceutical composition being capable of being administered at a rate of approximately 0.01 to approximately 20 mg/kg/injection.

The present invention also relates to a vaccine composition, characterized in that it comprises as active ingredient a monoclonal antibody as defined above, or an anti-idiotypic antibody as defined above, or Fab fragments as defined above, or an anti-anti-idiotypic antibody as defined above, in combination with a pharmaceutically acceptable adjuvant.

The present invention also relates to the use of an angiogenesis inhibitor, chosen from an antibody as defined above, an anti-idiotypic antibody as defined above, a Fab fragment as defined above, or a complex as defined above, or an anti-anti-idiotypic antibody as defined above, for preparing a medicament intended for the treatment of pathologies requiring inhibition of endothelial proliferation, in particular within the framework of the following pathologies: cancers, muscle degeneration linked with age, diabetic retinopathies, rheumatoid polyarthritis, angiomas, angiosarcomas, in particular Castelman's disease and Kaposi's sarcoma.

The present invention also relates to the use of an angiogenesis inhibitor, chosen from an antibody as defined above, an anti-idiotypic antibody as defined above, a Fab fragment as defined above, or a complex as defined above, or an anti-anti-idiotypic antibody as defined above, for preparing a medicament intended for the treatment of pathologies requiring inhibition of endothelial activation, in particular within the framework of the following pathologies: allograft and xenograft rejection, acrocyanosis, sclerodermas, or within the framework of the preparation of grafts between removal and transplantation.

The expression “inhibition of endothelial activation” can be determined by a test measuring the presence at the cell surface of an activation marker (in particular ICAM-1, V-CAM).

The present invention also relates to the use of an angiogenesis activator, in particular an antibody as defined above, an anti-idiotypic antibody as defined above, a Fab fragment as defined above, or an anti-anti-idiotypic antibody as defined above, for preparing a medicament intended to promote cicatrization in particular within the framework of injuries, tissue reconstruction in particular within the framework of muscle and cutaneous grafts during reconstructive and cosmetic surgery, ovarian induction, reperfusion of ischemic areas during arteritis of the lower limbs or myocardial infarction.

### DESCRIPTION OF THE FIGURES

FIGS. 1A, 1B, 1C and 1D represent the morphology of the two cell phenotypes in culture, namely the non-angiogenic endothelial cells of foetal bovine aorta (F/0) and the angiogenic endothelial cells of foetal bovine aorta (F/V).

FIG. 1A corresponds to F/0 cells cultured in the absence of VEGF

FIG. 1B corresponds to F/0 cells cultured in the presence of VEGF

FIG. 1C corresponds to F/V cells cultured in the absence of VEGF

FIG. 1D corresponds to F/V cells cultured in the presence of VEGF

It is noted that VEGF maintains distinct angiogenic phenotypes. Only the F/V cells change morphology under the action of VEGF. The F/O cells are rounded and larger than the F/V cells. The addition of VEGF does not modify the morphology of the F/O cells. The F/V cells cultured without VEGF have a morphology close to that of the F/O cells. In the presence of VEGF, they appear stretched.

FIGS. 2A, 2B and 2C represent the gene expression analysis by RNA transfer (Northern blot).

FIG. 2A corresponds to the comparison by RNA transfer of the expression of VEGF-R2 (VEGF receptor 2) in the F/0 and F/V cells. Actin, the expression of which does not vary in the F/0 and F/V cells, is used as control. This figure demonstrates that the expression of VEGF-R2 is strongly increased in the F/V cells relative to the F/0 cells.

FIG. 2B corresponds to the comparison by RNA transfer of the expression of collagen-1 in the F/0 and F/V cells. Actin, the expression of which does not vary in the cells is used as control. This figure demonstrates that the expression of collagen-1 is greater in the F/0 cells than in the F/V cells.

FIG. 2C corresponds to the comparison by RNA transfer of the expression of fibronectin in the F/0, F/V, BREC/0 (non-angiogenic endothelial cells of the retina) and BREC/V (angiogenic endothelial cells of the retina) cells (Hutchings et al., 2002). Actin, the expression of which does not vary in the cells is used as control. This figure demonstrates that the expression of fibronectin is greater in the F/0 cells than in the F/V cells. As for VEGF-R2, there is no significant difference in fibronectin expression in the BREC/V and BREC/0 cells.

In all these figures, the “ratio” line corresponds to the ratio between the expression of the gene of interest in the two cell types.

FIGS. 3A and 3B represent in vitro angiogenesis on Matrigel (Becton Dickinson).

FIG. 3A represents the absence of formation of tubules by the F/O cells. The F/O cells remain in an even monolayer on Matrigel.

FIG. 3B represents the formation of tubules by the F/V cells. The F/V cells differentiate on Matrigel, they are aligned end to end in order to reconstitute pseudo-capillaries.

FIG. 4 represents the sensitivity of the cells to particular anti-angiogenic agents, i.e.: ME-2, FUMA and SU5416.

The white columns correspond to the F/0 non-angiogenic endothelial cells and the black columns correspond to the F/V angiogenic endothelial cells.

“ME-2” corresponds to methoxyestradiol; “FUMA” corresponds to fumagillin; “SU 5416” is a VEGF-R2 kinase activity inhibitor.

The F/0 and F/V cells are identically sensitive to fumagillin (FUMA) and methoxyestradiol (ME-2). On the other hand, the F/V cells are much more sensitive than the F/0 cells to the anti-angiogenic agent interfering with VEGF-R2 (SU 5416).

FIGS. 5A, 5B and 6 illustrate the mitogenic functions of the monoclonal antibodies directed against the angiogenic endothelial cells. Certain antibodies are anti-angiogenic, i.e. capable of specifically inhibiting only the proliferation of the angiogenic endothelial cells (called type 3 antibodies). Other antibodies have a pro-angiogenic activity vis-à-vis endothelial cells (called type 5 antibodies).

FIGS. 5A and 5B show the anti-angiogenic action of the type 3 antibodies. FIG. 5A relates in particular to the action of the type 3 antibodies on the proliferation of the F/V cells of angiogenic phenotype. FIG. 5B relates to the action of the type 3 antibodies on the proliferation of the F/O non-angiogenic cells. The test carried out is a proliferation test as described in the examples. The cells are cultured in DMEM supplemented with 5% newborn calf serum to which is added either no angiogenic factor (condition 0), or 2 ng/ml VEGF (condition VEGF), or 2 ng/ml FGF-2 (condition FGF-2). The antibody is added at the same time as the angiogenic factors, at variable concentrations (0, 1, 3 or 7 μg/ml).

The white columns correspond to the proliferation of the cells without antibodies. The dotted columns correspond to the proliferation of the cells in the presence of 1 μg/ml of antibodies. The hatched columns correspond to the proliferation of the cells in the presence of 3 μg/ml of antibodies. The black columns correspond to the proliferation of the cells in the presence of 7 μg/ml of antibodies.

It is noted that the type 3 antibodies inhibit the proliferation of the F/V cells in a dose-dependent fashion, without affecting that of the F/0 cells.

It is noted that the type 3 antibodies inhibit the mitogenic effect of VEGF and of FGF-2 vis-à-vis angiogenic endothelial cells.

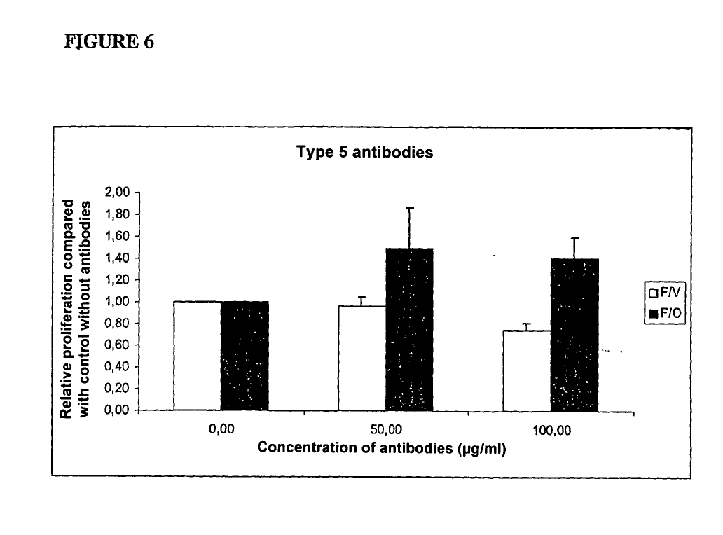

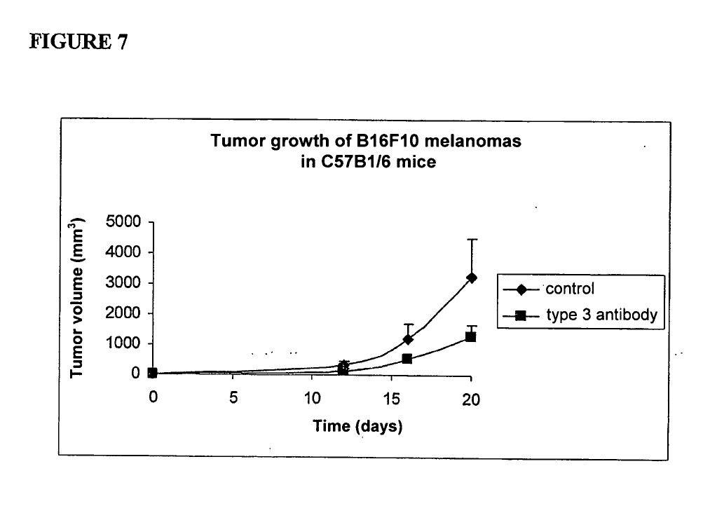

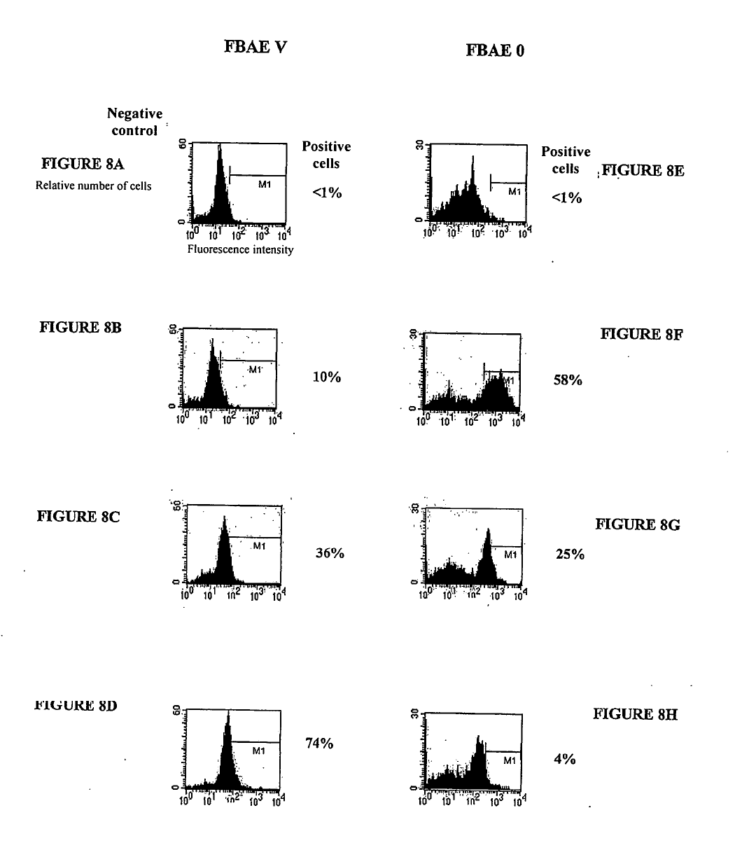

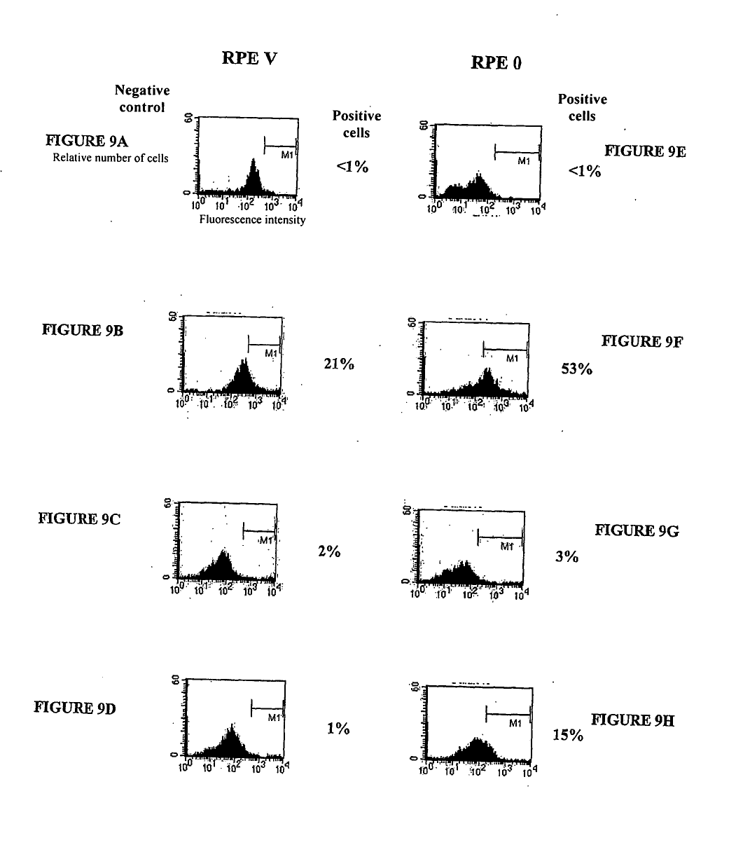

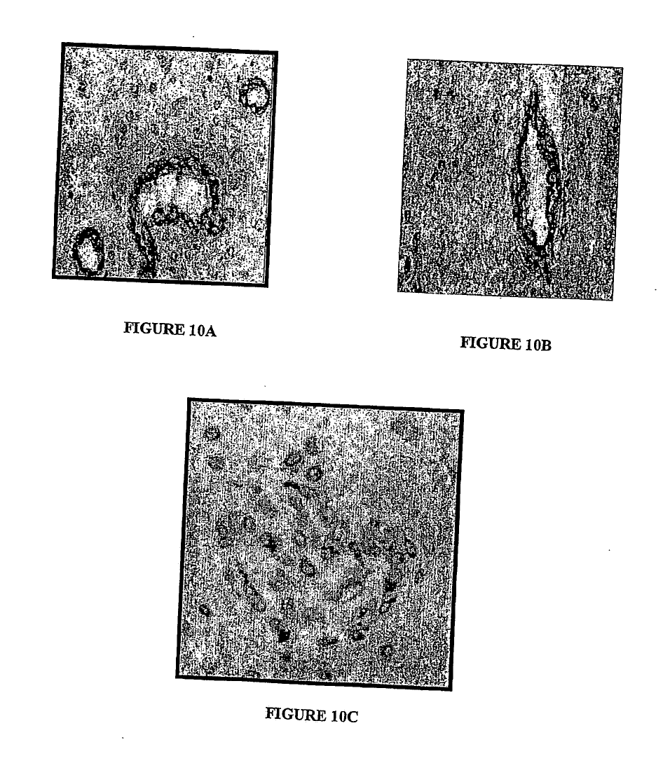

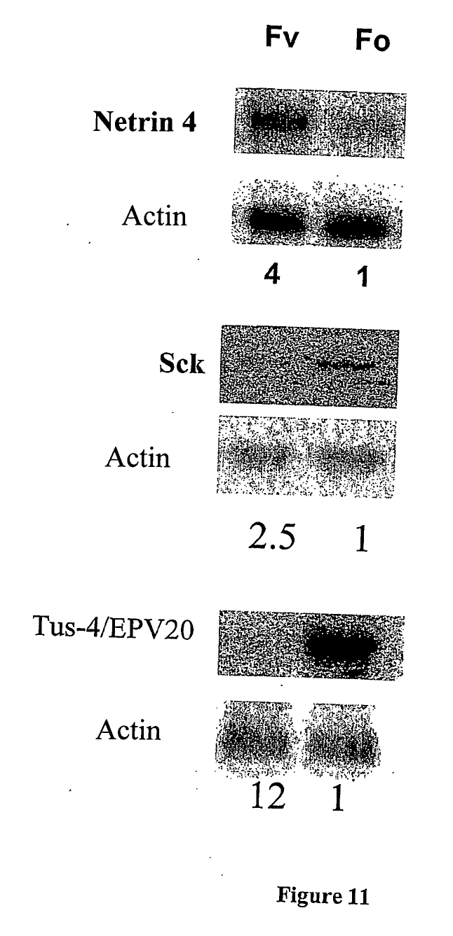

FIG. 6 shows the pro-angiogenic action of the type 5 antibodies. The test carried out is a proliferation test as described in the examples. The cells are cultured in DMEM supplemented with 5% newborn calf serum. The antibody is added at the same time as the angiogenic factors, at variable concentrations (0, 50 or 100 μg/ml).

The white columns correspond to the relative proliferation of the F/O cells in the presence of variable concentrations of type 5 antibodies. The black columns correspond to the relative proliferation of the F/V cells in the presence of variable concentrations of type 5 antibodies.

It is noted that the type 5 antibodies stimulate the proliferation of the F/O cells. On the other hand, they have no effect on the proliferation of the F/V cells.

FIG. 7 represents the function of the type 3 antibodies in tumorigenesis.

The curve with the black diamonds corresponds to untreated mice and the curve with the black squares corresponds to the type 3 antibodies.

This figure represents the average tumour volume (6 mice per group) in mm³ as a function of time (in days).

It is noted that the type 3 antibodies are capable of significantly inhibiting the development of B16F10 melanomas in C57BL/6 mice.

FIGS. 8A to 8H represent analyses by flow cytometry for F/V cells (FBAE V) (FIGS. 8A to 8D) and for F/0 cells (FBAE 0) (FIGS. 8E to 8H), incubated with different antibodies. FIGS. 8A and 8E correspond to the negative control; FIGS. 8B and 8F to the A antibody defined hereafter; FIGS. 8C and 8B to the B antibody defined hereafter; FIGS. 8D and 8H to the C antibody defined hereafter.

FIGS. 9A to 9H represent analyses by flow cytometry for RPE V cells (FIGS. 9A to 9D) and for RPE 0 cells (FIGS. 9E to 9H), incubated with different antibodies. FIGS. 9A and 9E correspond to the negative control; FIGS. 9B and 9F to the A antibody defined hereafter; FIGS. 9C and 9G to the B antibody defined hereafter; FIGS. 9D and 9H to the C antibody defined hereafter.

FIGS. 10A, 10B and 10C correspond to immunohistochemical results of glioblastoma sections. FIG. 10A corresponds to a glioblastoma incubated with an antibody recognizing the smooth muscle cells of the vessels (“SM actin”). FIG. 10B corresponds to a glioblastoma incubated with a commercial antibody recognizing endothelial cells (anti-CD 31 antibody) (DAKO). FIG. 10C corresponds to a glioblastoma incubated with an antibody recognizing the C antibody defined hereafter.

FIG. 11 corresponds to an RNA transfer, using probes corresponding to the 74/1268 fragment of the AF063658 sequence (Genbank) for VEGF-R2, to the 4155/4624 fragment of the AB008683 sequence (Genbank) for collagen-1 and to the 380/847 fragment of the M10905 sequence (Genbank) for fibronectin, to the AK024691 sequence of netrin 4, the AK 025662 sequence for Sck, the X85799 sequence for Tus-4/EPV20/.

### MATERIAL AND METHODS

1. Establishment of Clones with Different Phenotypes

Foetal bovine aorta endothelial (FBAE or F) cells are removed mechanically from the aortic lumen using a scraper and incubated in culture dishes previously lined with gelatin or collagen I (10 μg/ml) prepared according to the paragraph “in vitro angiogenesis” (0.2% in PBS, 24 hours at 4° C.) in DMEM medium containing 10% newborn calf serum (NCS). The dishes are either supplemented with oestradiol (10-9 M) and 1 ng/ml of VEGF (F/V) or cultured without oestrogen and without VEGF (F/0). The medium is changed after 4 days. Between 6 and 10 days, the clones of each of the cultures are isolated. For this purpose, clones visualized with the microscope as foci of 100 to 1000 cells are aspirated using a micropipette and transferred to 2 cm² wells. Each of the clones is cultured in the corresponding medium (oestradiol+VEGF for the F/V cells; without oestradiol and without VEGF for the F/0 cells) then once confluence is obtained (approximately 6 days for the F/V, 15 days for the F/0) the cell monolayers are trypsinated and reseeded in dishes 5 cm in diameter. Once confluence is obtained, the cells are again trypsinated, an aliquot fraction is frozen, then the validation (determination of the response to VEGF in one of the following tests: migration, proliferation, angiogenesis) of the clone is initiated.

Factors Used

VEGF (isoform of 165 amino acids) is produced by infection of Sf9 insect cells by a recombinant baculovirus containing the corresponding cDNA (Plouet et al., 1997).

2. In Vitro Study Techniques: Validation of the Clones

Migration

The technique has been described previously by Jonca et al. (1997).

Proliferation

12-well plates are seeded at a low density (5000 to 10,000 cells/well), in DMEM medium with 8% serum. After 24 hours, the proliferation of the cells is stimulated by the addition of a range of growth factor, anti-angiogenic medicaments (methoxyestradiol, fumagillin, SU5416 targeted on VEGF-R2—Plouët et al., 2001) or antibodies of the present invention. The cells are restimulated after 48 hours and then after a total of five days in culture the cells are trypsinated and counted with a Coulter counter.

In Vitro Angiogenesis

Four rat tails were skinned and dissected in order to recover the white bundles which are mainly constituted by type I collagen. The collagen is extracted from these fibres in 50 ml of cold 0.5 M acetic acid and stirred overnight. The liquid is then centrifuged at 5000 g for 40 minutes and the supernatant is recovered. The extraction is repeated once with 20 ml of acetic acid, the supernatants are mixed and then dialyzed against 1 l of 0.2 M acetic acid. The collagen concentration is adjusted to 3 mg/ml by weighing.

The preparation of the gels for the in vitro angiogenesis is carried out on ice in order to preserve the collagen solution in liquid form. One ml of collagen (5 mg/ml) is mixed with 0.5 ml of 10×DMEM (containing a 10× concentration of antibiotics and glutamine), 0.9 ml of sterile H₂O and 0.1 ml of 1M sodium bicarbonate. Once the pH is adjusted to 7.4, an equal volume of matrigel (Becton Dickinson) is added.

The gel is poured into culture wells (2 mm thick) and incubated at 37° C. in order to solidify. The cells are added (100,000 cells/cm²) onto the surface of the gel 15 minutes later. After 2 hours, the different soluble factors are added and the cells are observed and photographed after 24 hours.

RNA Transfer (Northern Blot)

The F/0 or F/V cells are cultured to semi-confluence in DMEM medium containing 10% NCS. The cells are then trypsinated and centrifuged. The RNAs are extracted using a solution of TRIZOL (Invitrogen). 2 μg of poly A+RNA are separated by electrophoresis in 1.2% agarose gel (p/vol) under denaturing conditions, transferred onto a nylon membrane (Hybond N Amersham) and fixed under UV (0.5 J/cm2) according to the technique described by Sambrook et al. in 1989. Hybridization is carried out at 42° C. in the following reaction medium: 50% formamide, 5× Denhardt's and 0.5% SDS (w/vol), containing 0.2% herring sperm DNA and 1 ng/ml radioactive probe (specific activity comprised between 10⁸ and 10⁹ cpm/μg) labelled with 32P using the Megaprime™ DNA labelling systems kit (Amersham). Washings are carried out at 45° C. in 0.2×SSPE containing 0.1% SDS.

The probes used correspond to the 74/1268 fragment of the AF063658 sequence (Genbank) for VEGF-R2, the 4155/4624 fragment of the AB008683 sequence (Genbank) for collagen-1 and the 380/847 fragment of the M10905 sequence (Genbank) for fibronectin, to the AK024691 sequence of netrin 4, the AK 025662 sequence for Sck, the X85799 sequence for Tus-4/EPV20/

3. Production of Monoclonal Antibodies Specific to the FBAE V Phenotype

Subtractive Immunization of Mice by FBAE Cells

Five 6-week-old Balb/c mice are immunized by a 200-μl injection containing 2×10⁶ F/0 cells. Twenty-four and forty-eight hours later, the mice receive 200 mg/kg of body weight of cyclophosphamide in order to destroy the active lymphocytes and thus render the mice tolerant to the endothelium with a non-angiogenic phenotype. Fifteen days later, the mice receive an injection of 2×10⁶ F/V cells. The operation is repeated on day 30 and on day 45. The injections were carried out by intraperitoneal route. The response of each mouse to the immunization is evaluated from a serum sample by flow cytometry.

Fusion

The technique used for producing the hybridomas is derived from that developed by Kohler and Milstein (1975).

The day before the fusion, i.e. on Day 47, five non-immunized Balb/c mice are sacrificed in order to obtain the macrophages serving as feeder cells to the hybridomas. The macrophages are recovered by injection of 8 ml of ISCOVE's culture medium into the peritonea of the mice. The medium is aspirated and returned twice into the peritoneal cavity then recovered and centrifuged. After centrifugation, the macrophages are taken up in ISCOVE's medium containing 20% fetal calf serum (Biochrom, lot 5612). These cells are then distributed into 20 96-well plates at a rate of 100 μl per well (approximately 3×10⁴ cells/well).

The next day, i.e. on Day 48, the mice having the highest titres of antibodies directed against the F/V cells are sacrificed. Their spleens are removed and dilacerated in ISCOVE's medium in order to release the splenocytes. The conjunctive tissue is released and the splenocytes are centrifuged and counted. In parallel, the Ag8X63 mouse myeloma line was cultured for 10 days in ISCOVE's medium (Invitrogen) with 20% Myoclone serum (Invitrogen). These cells are washed and counted.

The two cell types, splenocytes and myelomas, are mixed in order to obtain a ratio of 1 Ag8X63 myeloma cell (Kearney et al., 1979) per 6 splenocytes. The fusion is carried out by the addition of 20 times 50 μl of polyethylene glycol (PEG) at 30-second intervals. Four ml of ISCOVE's medium preheated to 37° C. are then added dropwise to the cell suspension, then after an incubation period of 4 minutes at 37° C., 4 ml are added. The suspension is centrifuged then the cell pellet is taken up in 100 ml of ISCOVE's medium complemented with 20% fetal calf serum and HAT 1× selective medium (50×: 5 mM Hypoxanthine, 20 μM Aminopterin and 0.8 mM Thymidine) which allows the elimination of the splenocytes and myeloma cells. The suspension is then distributed at a rate of 100 μl per well onto the macrophages (approximately 100,000 cells).

After 5 days, 100 μl of HAT medium are added, and between 8 and 14 days the conditioned medium of each hybridoma is samlped in order to measure the antibodies directed against the endothelial cells with different phenotypes by ELISA.

The hybridomas selected by their capacity to secrete antibodies directed against the F/0 or F/V cells are then cloned (first cloning), i.e. the cells are seeded under limited dilution conditions (5 cells/ml) in a volume of 0.1 ml per well. The medium (ISCOVE supplemented with serum and HAT) is changed after 10 days. After 15 days, some wells contain foci of cells which have multiplied from the cell seeded at the start, therefore all these cells are identical and originate from the same clone. When the surface occupied by the cells represents at least half of the total surface of the well, the medium is sampled and analyzed as previously. At this stage the antibody prodcuing clones can be selected and their specificity for the F/0 or F/V cells can be known (second cloning).

Once the clones have been identified, their monoclonal nature is affirmed by the standard operation consisting in seeding a 96-well plate with cells originating from the same clone diluted under limited conditions as previously. The secreting clones must therefore all secrete an antibody of the same specificity vis-à-vis F/V cells in comparison with F/0 cells (screening by ELISA) in order for this antibody to be declared monoclonal.

A third cloning is then carried out in order to ensure that the clones are indeed monoclonal under conditions identical to those indicated above.

Method for Screening Clones

The screening of the serum sampled and of the supernatants of the hybridomas is carried out by ELISA and flow cytometry.

ELISA

15,000 F/0 or F/V cells are seeded per well in 96-well plates (100 μl/well) in DMEM medium containing 10% newborn calf serum. After 36 hours, the sub-confluent cells are rinsed 3 times in a solution of PBS containing 0.005% Tween 20. The potential binding sites not specific to the antibodies are saturated by incubation in a solution of 1 M glycine, pH 8.1 (2 hours at ambient temperature) followed by 3 washings (PBS/0.05% Tween) and incubation overnight at 4° C. in a 10 μg/ml solution of irrelevant IgG in PBS. The blocking solution is eliminated, then the antibodies to be tested are added. After incubation for 2 hours at ambient temperature, the cells are washed 3 times in PBS/0.05% Tween in order to eliminate the non-fixed antibodies. The cell-antibody complexes are incubated for 1 hour in the presence of an anti-mouse secondary antibody coupled with peroxidase (Amersham NA 931) diluted 1/4000 in PBS containing 0.05% Tween and 0.5% BSA. After 3 washings (PBS/0.05% Tween), peroxidase substrate (OPD Sigma) is added at a rate of 100 μl/well. After incubation for 10 to 30 minutes sheltered from the light, the calorimetric reaction is stopped by the addition of 100 μl of H₂SO₄M. The intensity of the signal is measured by spectrophotometry at 490 nm.

Cytometry

The supernatant (or the serum) to be tested is incubated with F/0, F/V, RPE/0 cells or RPE/V cells for 45 minutes. The cells are then washed 3 times in PBS-BSA 0.02%. A second mouse anti-IgG antibody coupled with fluorescein isothiocyanate is added to the cells (F-877, Sigma Immuno Chemicals). The fluorescence intensity of the cells is measured by flow cytometry and compared between the F/0 and F/V cells. The fluorescence intensity is measured in an ELITE flow cytometer.

Isotyping

The isotype characterization of each clone is measured using a MABTEST murine antibodies (Adiatec) rapid isotyping test.

Tumorigenesis

200,000 B16 melanoma cells are injected into C57 mice. Eight days later, when the tumours have produced palpable nodules, the antibodies are injected twice a week by intraperitoneal route at a dose of 50 μg diluted in 200 μl of PBS. The volume of the tumour is measured twice a week with skinfold calipers and expressed by the formula V=½×L×l² (L and l respectively designating the largest and smallest dimension).

4. Production of Anti-Idiotypic Antibodies

Mice are injected by sub-cutaneous route with 10 μg of a monoclonal antibody directed against non-angiogenic endothelial cells, said monoclonal antibody being obtained according to the process described in the previous paragraph, in combination with 100 μl of Freund's adjuvant (Sigma), the antibodies being purified according to standard methods by precipitation with ammonium sulphate then by ion-exchange chromatography.

The injection is repeated 15, 30 and 45 days later.

Fifty-five days after the first injection, mice are injected with 10 μg of the same antibodies by intraperitoneal route. Fifty-eight days later, the mice are sacrificed and their spleens are removed and dilacerated in ISCOVE's medium in order to release the splenocytes. The splenocytes are fused with mouse myeloma cells, in particular AG8X 63 cells (Kearney et al., 1979) and incubated at a rate of 100,000 cells/well.

The fusion is carried out by the addition of 20 times 50 μl of polyethylene glycol (PEG) at 30-second intervals. Four ml of ISCOVE's medium preheated to 37° C. are then added dropwise to the cell suspension, then after an incubation period of 4 minutes at 37° C., 4 ml are added. The suspension is centrifuged then the cell pellet is taken up in 100 ml of ISCOVE's medium complemented with 20% fetal calf serum and HAT 1× (50×: 5 mM Hypoxanthine, 20 μM Aminopterin and 0.8 mM Thymidine) and distributed onto the macrophages at a rate of 100 μl per well.

After 5 days, 100 μl of HAT medium are added, and between 8 and 14 days the conditioned medium of each hybridoma is removed in order to measure the antibodies directed against the antibodies having served as immunogenic agents, i.e. directed against endothelial cells.

The hybridomas selected by their ability to secrete antibodies directed against monoclonal antibodies themselves directed against F/0 or F/V cells are then cloned (first cloning), i.e. the cells are seeded under limited-dilution conditions (5 cells/ml) under a volume of 0.1 ml per well. The HAT culture medium is changed after 10 days. After 15 days, certain wells contain foci of cells which have multiplied from the cell seeded at the start, therefore all these cells are identical and originate from the same clone. When the surface occupied by the cells represents at least half of the total surface of the well, the medium is removed and analyzed as previously by ELISA. At this stage the clones producing antibodies can be selected and their specificity for the monoclonal antibodies directed against the F/0 or F/V cells can be known (second cloning).

Once the clones have been identified, their monoclonal nature is affirmed by the standard operation consisting of seeding a 96-well plate with cells originating from the same clone diluted under limited conditions as previously. The secreting clones must therefore all secrete an antibody of the same specificity in order for this antibody to be declared monoclonal.

A third cloning is then carried out in order to ensure that the clones are indeed monoclonal under conditions identical to those defined above.

The anti-idiotypic antibodies are screened by a battery of tests, in particular by an anti-idiotypic ELISA test: the antibodies directed against the endothelial cells of the invention are placed in 50 mM carbonate buffer, pH 9, at a dilution of 10 μg/ml. The surface of the well is then incubated with 0.5% serum albumin. The hybridoma supernatants are then incubated in dishes and their presence is developed by the mouse anti-immunoglobulin antibodies conjugated with peroxidase.

It is also verified that the anti-idiotypic antibodies inhibit the function of the monoclonal antibodies against which they are directed vis-à-vis endothelial cells, in particular that they inhibit the proliferation of the F/V cells incubated with ng/ml and 20 μg/ml of angiogenic or non-angiogenic endothelial anti-cell monoclonal antibodies.

5. Production of Anti-Anti-Idiotypic Antibodies

Mice are injected by sub-cutaneous route with 10 μg of an anti-idiotypic antibody, such as obtained according to the process described in the previous paragraph, in combination with 100 μl of Freund's adjuvant.

The injection is repeated 15, 30 and 45 days later.

Fifty-five days after the first injection, mice are injected with 10 mg of anti-idiotypic antibodies by intraperitoneal route. Fifty-eight days later, the mice are sacrificed and their spleens are removed and dilacerated in ISCOVE's medium in order to release the splenocytes. The splenocytes are fused with mouse myeloma cells, in particular AG8X63 cells, and incubated at a rate of 100,000 cells/well.

The fusion is carried out by the addition of 20 times 50 μl of polyethylene glycol (PEG) at 30-second intervals. Four ml of ISCOVE's medium preheated to 37° C. are then added dropwise to the cell suspension, then after an incubation period of 4 minutes at 37° C., 4 ml are added. The suspension is centrifuged then the cell pellet is taken up in 100 ml of ISCOVE's medium complemented with 20% fetal calf serum and HAT 1× (50×: 5 mM Hypoxanthine, 20 μM Aminopterin and 0.8 mM Thymidine) and distributed at a rate of 100 μl per well onto the macrophages.

After 5 days, 100 μl of HAT medium are added, and between 8 and 14 days the conditioned medium of each hybridoma is sampled in order to measure the antibodies directed against the anti-idiotypic antibodies.

The hybridomas selected by their ability to secrete antibodies directed against anti-idiotypic antibodies are then cloned (first cloning), i.e. the cells are seeded under limited-dilution conditions (5 cells/ml) under a volume of 0.1 ml per well. The ISCOVE's culture medium containing 20% serum and HAT is changed after 10 days. After 15 days, some wells contain foci of cells which have multiplied from the cell seeded at the start, therefore all these cells are identical and originate from the same clone. When the surface occupied by the cells represents at least half of the total surface of the well, the medium is sampled and analyzed as previously by ELISA. At this stage the antibody producing clones can be selected and their specificity for the anti-idiotypic antibodies can be known (second cloning).

Once the clones have been identified, their monoclonal nature is affirmed by the standard operation consisting of seeding a 96-well plate with cells originating from the same clone diluted under limited conditions as previously. The secreting clones must therefore all secrete an antibody of the same specificity in order for this antibody to be declared monoclonal.

A third cloning is then carried out in order to ensure that the clones are indeed monoclonal, identical to those described above.

The anti-anti-idiotypic antibodies are screened by a battery of tests, in particular by an anti-idiotypic ELISA test: the anti-idiotypic antibodies as previously obtained are placed in 50 mM carbonate buffer, pH 9, at a dilution of 10 μg/ml. The surface of the well is then incubated with 0.5% serum albumin. The hybridoma supernatants are then incubated in dishes and their presence is developed by mouse anti-immunoglobulin antibodies conjugated with peroxidase.

It is also verified that the anti-anti-idiotypic antibodies bind to the angiogenic or non-angiogenic endothelial cells, according to the same tests as those described in paragraph 3.

6. Immunohistochemistry of Glioblastoma Sections

Glioblastoma is a malign tumour of the cerebrum, the major characteristic of which is to be extremely vascularized. Classified sections of pieces of glioblastomas removed during surgical operations were incubated with antibodies recognizing the smooth muscle cells of vessels (SM actin) (FIG. 10A) or the endothelial cells (anti-CD 31) (FIG. 10B) or the C antibody defined above (FIG. 10C).

Sections of glioblastomas with a thickness of 3 μm are deparaffinated.

Then, the antigenic sites are exposed in the following fashion: said sections are subjected to 2 5-minute micro-wave passes (maximum power) in 10 mM Tp Citrate, pH 6.0 (tri-sodium citrate C₆H₅Na₃O₇, 2H₂O MW 294.10), and the slides are left to cool down at ambient temperature for 30 minutes then placed to wait in PBS 1×.

The 10 mM citrate buffer is obtained as follows: 5.9 g of tri-sodium citrate and water (qs 2 l) are mixed then the pH is adjusted to 6 with citric acid in powder form.

The non-specific sites are blocked by being brought into the presence of the abovementioned sections with a prediluted blocking serum (2.5% horse serum) for 20 minutes. Then the excess serum is eliminated by rinsing in PBS for 5 minutes.

The sections are incubated for 30 minutes with different antibodies (diluted in PBS containing 0.1% BSA), at ambient temperature, in a humid chamber, and rinsed with PBS for 5 minutes.

An additional incubation is carried out with universal biotinylated secondary antibodies prediluted for 30 minutes (VECTASTAIN Elite UNIVERSAL RTU KIT).

The mixture thus obtained is incubated with the Elite ABC avidin-biotin-peroxidase complex for 30 minutes, then rinsing is carried out with PBS for 5 minutes.

Incubation is carried out with ready-to-use AEC reagent (Dako K3464) for 10 minutes, followed by rinsing with distilled water.

Then counter-colouring is carried out with Mayer haematoxylin for 2 minutes followed by rinsing with ammoniated water (2.5/1000) then with distilled water.

The slide is then incubated with aqueous mounting liquid (Dako faramount S3025) and a microscope cover glass put into position.

Results

I. Determination of the Phenotypes of the F/O and F/V Cells.

Morphology

The phenotype of the F/0 and F/V strains is maintained by the addition of VEGF to the long term culture medium. When the VEGF is removed (case of the absence of VEGF), the morphology of each of the strains is different: pavimentous even monolayer at confluence for F/V, the F/0s are larger and more rounded cells.

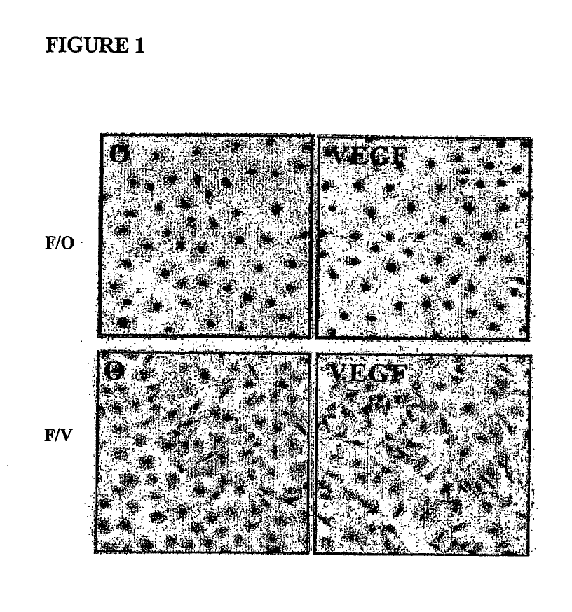

Four hours after the addition of the VEGF, only the F/V cells undergo a change in morphology: the cells become elongated, the intercellular space increasing (see FIGS. 1C and 1D). On the other hand, the F/0 cells do not change shape (FIGS. 1A and 1B).

Expression of Genes

The expression of VEGFR-2 and collagen I was studied in F/0 and F/V cells by Northern Blot. It appears that the expression of VEGFR-2 is increased 4-fold in the F/V cells in comparison with F/0. On the other hand the expression of VEGFR-2 is identical in BREC/0 and BREC/V (Hutchings et al., 2002).

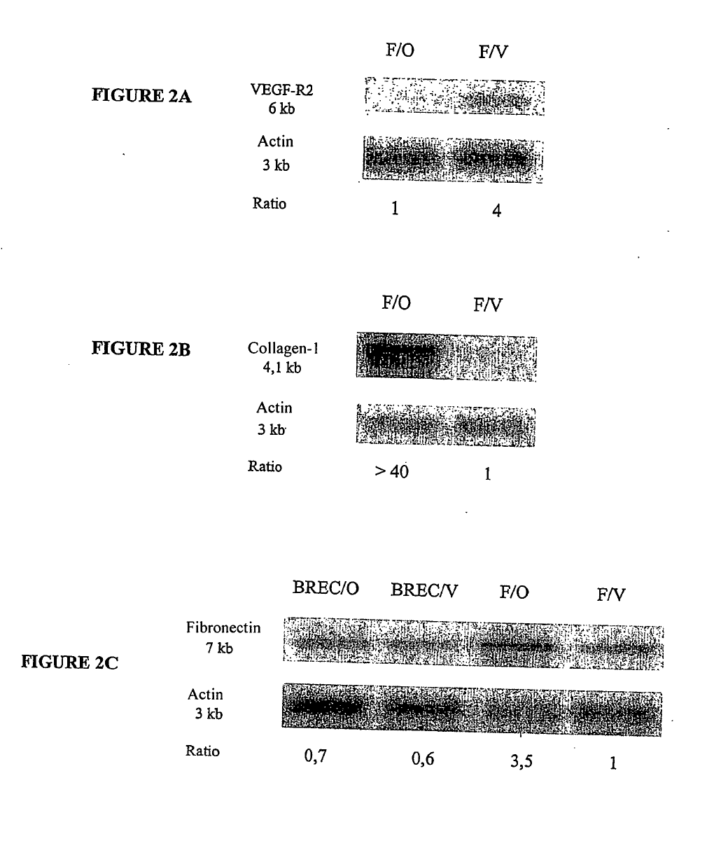

Moreover, the expression of other genes was compared in the 0 and V pairs of bovine retinal endothelial cells (BREC) and bovine fetus endothelial cells (F) (see FIG. 2C).

For example, fibronectin appears more expressed by the F/0 cells than by the F/V cells (3.5 ratio) whereas its expression is similar in the BREC/0 and BREC/V cells.

For example the sequences AK024691, AK 025662, and X85799 are respectively 4, 12 and 2.5 times more expressed in the FV cells than the F/0 cells

In Vitro Angiogenesis in Response to VEGF

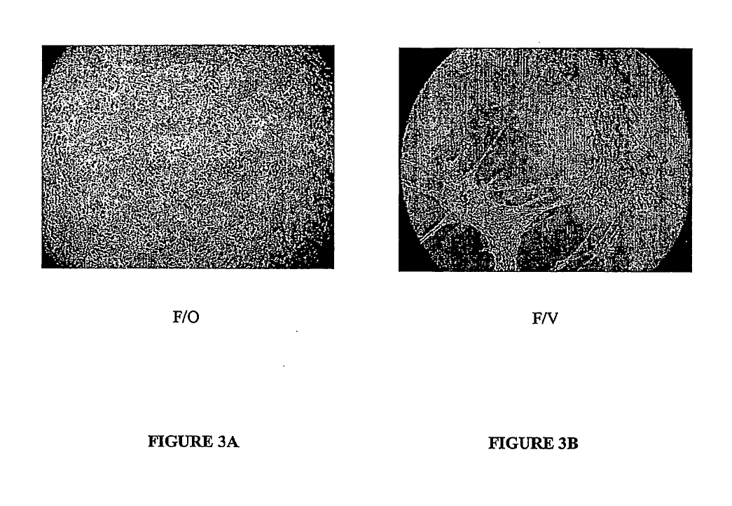

The F/0 or F/V cells seeded on a collagen-matrigel gel are incubated at 50 ng/ml of VEGF and their morphology is examined 24 hours later. The F/V cells stimulated by the VEGF organize themselves into bundles comprising a row of 3-4 cells then into tubular structures as shown by FIG. 3B. On the other hand the F/0 cells remain arranged in clusters, whether or not they are stimulated by VEGF (FIG. 3A).

Response to the Anti-Angiogenic Treatments

The F/V cells are sensitive to the mitogenic action of VEGF (ED 50=0.3 ng/ml) whereas the F/0 cells do not proliferate under the action of VEGF.

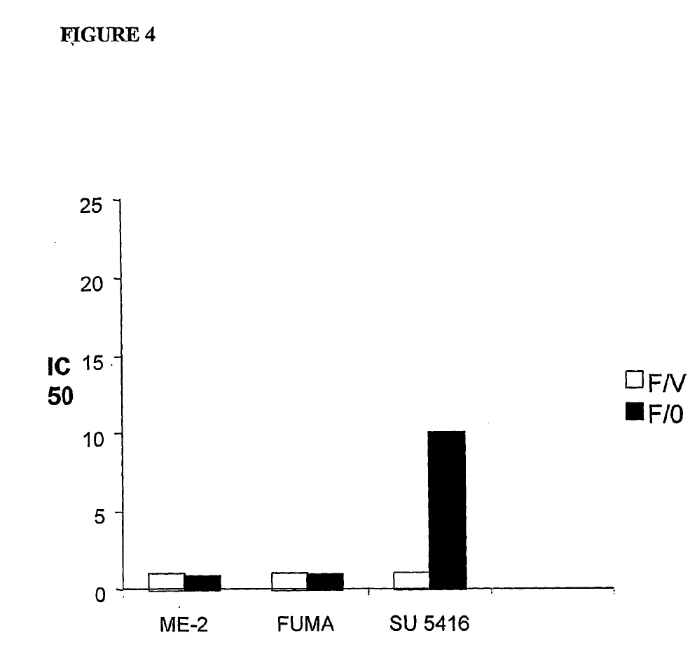

The F/0 and F/V cells are seeded at a low density (5000) as indicated and three drugs are inoculated separately, 2 of which are of unknown mechanism (Methoxyestradiol-ME-2 and fumagillin—FUMA) and 1 interfering with VEGF-R2 (SU 5416) inhibiting kinase activity. It appears that the F/0 and F/V cells are equally sensitive to fumagillin and methoxyestradiol (FIG. 4) whereas the F/V cells are more sensitive to SU 5416. It appears that the IC50 of these 2 compounds is respectively multiplied by 20 and 50 for the F/0 cells.

Thus the use of these cells makes it possible to carry out a screening of anti-angiogenic medicaments which do not affect the endothelial cells of healthy, non-angiogenic vessels.

II. Production of Monoclonal Antibodies Specific to the Angiogenic Phenotype

Specific Recognition of F/V Cells

4 fusions were carried out and 426 hybridomas were obtained. After screening by ELISA, 15 clones were sub-cloned and 1026 clones analyzed, 6 of which were selected by ELISA, corresponding to monoclonal antibodies binding to F/0 or F/V. Verification of the specificity of these monoclonal antibodies was carried out by flow cytometry.

It appears that certain antibodies bind selectively to F/V cells or F/0 cells (see table below and FIG. 8 representing the analysis of the specificity of the antibodies by flow cytometry).

The table below indicates the percentages of cells recognized by the different types of antibodies.

| type of | | | | | |
|---|---|---|---|---|---|
| antibodies | F/0 | F/V | BREC/0 | BREC/V | muscle |
| A | 58 | 10 | 44.7 | 95.9 | 5.4 |
| B | 25 | 36 | 4.2 | 8.4 | 0 |
| C | 3 | 48 | 43 | 63.7 | 3.8 |

3 types of antibodies can be distinguished:
Labelling F/0 > F/V: type A
Labelling F/V > F/0: type B
Labelling F/O absent: type C

The A antibody preferentially labels F/0 (FIG. 8 F) and RPE 0 cells (FIG. 9 F).

The B antibody preferentially labels F/V cells (FIG. 8 C), and does not recognize RPE 0 and RPE V.

The C antibody exclusively labels F/V cells (FIG. 8 D) and not F/0 cells (FIG. 8 H) and does not recognize RPE 0 and RPE V.

Proliferation

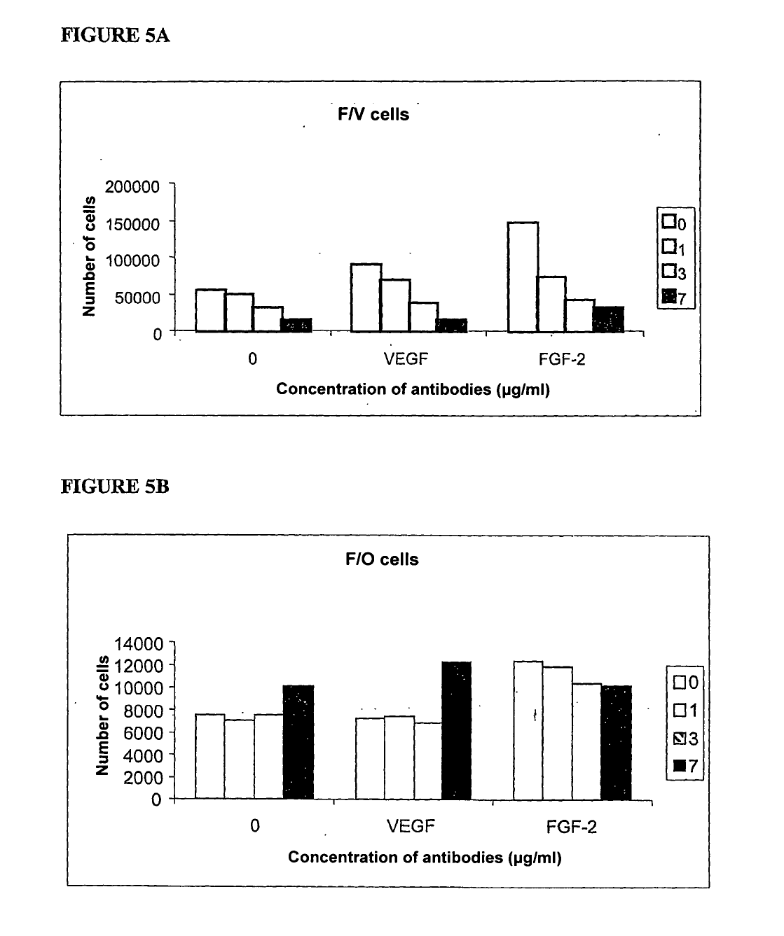

Study of the mitogenic function of the antibodies makes it possible to classify them in 2 categories: type 3 antibodies inhibit the proliferation of the F/V cells without affecting that of the F/0 cells (FIGS. 5A and 5B) and type 5 antibodies stimulate the proliferation of the F/0 cells without affecting that of the F/V cells (FIG. 6).

Tumorigenesis

FIG. 7 shows that the injection of the type B antibodies reduces the volume of the tumour at each observation time. The following table shows the averages and the standard deviations of the volumes of the tumours observed at 20 days. It is deduced from this that type A antibodies have no effect on tumorigenesis, which is in accordance with the results of flow cytometry demonstrating that this antibody does not bind to angiogenic cells.

On the other hand type B and C antibodies recognizing cells with an angiogenic phenotype by flow cytometry inhibit tumour growth.

| | | standard |
|---|---|---|
| Injection | average (mm3) | deviation (mm3) |
| PBS | 3226 | 1255 |
| Antibody A | 2159 | 350 |
| Antibody B | 1299 | 368 |
| Antibody C | 1866 | 287 |

III. Immunohistochemistry of Glioblastoma Sections

FIGS. 10A, 10B and 10C show
labelling with anti-actin antibodies (“smooth muscle cell actin”): labelling of all the smooth muscle perivascular cells;
labelling with anti-CD 31 antibodies: labelling of all the endothelial cells; and
labelling with C antibodies: labelling of some tumorous endothelial cells.

Moreover, labelling with C antibodies has never been detected in sections of healthy tissue (cerebrum, lung, heart, kidney, liver).

In conclusion the abovementioned antibody is therefore specific to cells with an angiogenic phenotype and does not recognize cells with a non-angiogenic phenotype.

### REFERENCES

Battegay, E. J., Rupp, J., Iruela-Arispe, L., Sage, E. H., Pech, M. (1994) PDGF-BB modulates endothelial proliferation and angiogenesis in vitro via PDGF b-receptors. J. Cell. Biol. 125, 917-928.
Boudreau, N., Andrews, C., Srebrow, A., Ravanpay, A., Cheresh, D. A. (1997) Induction of the angiogenis phenotype by Hox D3. J. Cell. Biol. 139, 257-264.
Brooks, P. C., Lin, J., French, D. L., Quigley, J. P. (1993) Subtractive immunization yields monoclonal antibodies that specifically inhibit metastasis. J. Cell. Biol. 122, 1351-1359.
Brooks P C., Clark R A., Cheresh D A. (1994) Requirement of vascular integrin alpha v beta 3 for angiogenesis. Science 264, 569-71.
Concina P, Sordello S., Elhage N., Fournial P., Plouët J., Bayard F., Arnal J-F. (2000) The mitogenic effect of 17 β-Estradiol on in vitro endothelial cell proliferation and on in vivo reendothelialization are both dependent on VEGF. J. Vasc. Res. 37, 202-208.
Gavrieli Y., Sherman Y., Ben-Sasson S A. (1992) Identification of programmed cell death in situ via specific labeling of nuclear DNA fragmentation. J. Cell. Biol. 119, 493-501.
Hutchings, H., Maitre-Boube, M., Tomban-Tink, J., Plouët, J. (2002) Pigment epithelium-derived factor exerts opposite effects on endothelial cells of different phenotypes. BBRC 294, 764-769.
Jonca F., Ortéga N., Gleizes P.-E., Bertrand N., Plouët J. (1997) Cell release of bioactive fibroblast growth factor by exon 6 encoded sequence of vascular endothelial growth factor. J. Biol. Chem. 272, 24203-24209.
Kearney J F., Radbruch A., Liesegang B., Rajewsky K. (1979) A new mouse myeloma cell line that has lost immunoglobulin expression but permits the construction of antibody-secreting hybrid cell lines. J. Immunol. 123, 1548-50.
Kohler et Milstein (1975) Continuous cultures of fused cells secreting antibody of predefined specificity. Nature, August 7; 256(5517): 495-7.
Plouët J., Moro F., Bertagnolli S., Coldeboeuf N., Mazarguil H., Clamens S., Bayard F. (1997) Extracellular cleavage of the vascular endothelial growth factor 189-amino acid form by urokinase is required for its mitogenic effect. J Biol. Chem. 272, 13390-96.
Plouët et al. (2001) Angiogenesis, 3, 215-217
Sambrook K., Fritsch E, Maniatis T. (1989) “Molecular cloning: a laboratory manual” Cold Spring Harbor ed. (C. S. H. Laboratory, Ed.) New York.

1-26. (canceled)

27. A method for the screening of angiogenic substances vis-à-vis endothelial cells with a non-angiogenic phenotype, not substantially affecting endothelial cells with an angiogenic phenotype, or
of anti-angiogenic substances vis-à-vis endothelial cells with an angiogenic phenotype, not substantially affecting endothelial cells with a non-angiogenic phenotype, comprising screening said substances with a binary assembly comprising endothelial cells with a non-angiogenic phenotype and endothelial cells with an angiogenic phenotype.

28. A method for the screening of anti-angiogenic substances vis-à-vis endothelial cells with an angiogenic phenotype, not substantially affecting endothelial cells with a non-angiogenic phenotype, comprising screening said substances with a binary assembly comprising endothelial cells with a non-angiogenic phenotype and endothelial cells with an angiogenic phenotype.

29. A process for screening a substance capable of inhibiting the angiogenesis of endothelial cells with an angiogenic phenotype without substantially affecting endothelial cells with a non-angiogenic phenotype, comprising the steps of:
preparing a culture of endothelial cells with an angiogenic phenotype;
preparing a culture of endothelial cells with a non-angiogenic phenotype;
adding to each of said cultures a mitogenic factor selected from the group consisting of FGF, PDGF, VEFG and EGF, and said substance capable of inhibiting angiogenesis;
maintaining each of said cultures with said added mitogenic factor and said substance for a time sufficient for at least one cell division cycle to occur;
comparing the inhibition of mitogenic action of said mitogenic factor by said substance on said endothelial cells with an angiogenic phenotype to said endothelial cells with a non-angiogenic phenotype.

30. A binary assembly comprising:
endothelial cells with a non-angiogenic phenotype comprising at least one of the following properties:
said cells remain confluent in the presence of growth factor VEGF without forming tubules,
said cells do not proliferate under the action of VEGF, and
said cells are not protected from apoptosis by VEGF;
and endothelial cells with an angiogenic phenotype comprising at least one of the following properties:
said cells form tubes in the presence of growth factor VEGF in a collagen gel,
said cells proliferate under the action of VEGF, and
said cells are protected from apoptosis by VEGF.

31. Endothelial cells with a non-angiogenic phenotype comprising at least one of the following properties:
said cells remain confluent in the presence of growth factor VEGF without forming tubules,
said cells do not proliferate under the action of VEGF, and
said cells are not protected from apoptosis by VEGF, or
an angiogenic phenotype comprising at least one of the following type of properties:
said cells form tubes in the presence of growth factor VEGF in a collagen gel,
said cells proliferate under the action of VEGF, and
said cells are protected from apoptosis by VEGF.

32. The endothelial cells according to claim 31,
characterized in that these are endothelial cells of the aorta, adrenal cortex, skin, cerebrum, retina, veins or umbilical cord artery.

33. A process for preparing endothelial cells according to claim 31, comprising the steps of:
incubating endothelial cells in a medium containing oestradiol and a growth factor to obtain clones of endothelial cells;
removing a clone using a micropipette, from said clones of endothelial cells;
preparing a culture of said clone;
maintaining said culture of said clone until cell confluence is obtained;
identifying endothelial cells from said culture of said clone using proliferation, migration or in vitro angiogenesis tests.

34. A polyclonal or monoclonal antibody directed against endothelial cells according to claim 31.

35. A process for preparing a monoclonal antibody according to claim 34 that is capable of activating angiogenesis comprising the steps of:
immunizing an animal by injection of cells with an angiogenic phenotype;
fusing myelomas of an animal and splenocytes of an animal in order to obtain hybridomas;
preparing a culture of said hybridomas;
cloning of said hybridomas and secreting antibodies against cells with an angiogenic phenotype; and
verifying angiogenesis-activation properties of said antibodies vis-à-vis angiogenic cells.

36. A process for preparing a monoclonal antibody according to claim 34 that is capable of inhibiting angiogenesis comprising the steps of:
immunizing an animal by injection of cells with an angiogenic phenotype;
fusing myelomas of an animal and splenocytes of an animal in order to obtain hybridomas;
preparing a culture of said hybridomas;
cloning of said hybridomas and secreting antibodies against cells with an angiogenic phenotype; and
verifying angiogenesis-inhibiting properties of said antibodies vis-à-vis angiogenic cells.

37. Anti-idiotypic antibodies directed against antibodies according to claim 34.

38. A Fab fragment of the monoclonal or polyclonal antibodies according to claim 34, or an anti-idiotypic antibody raised against said antibodies, said fragments being capable of activating or inhibiting angiogenesis.

39. A complex between:
an antibody according to claim 34 or a Fab fragment of said antibody, angiogenesis activator, and
a radioactive element containing an ionizing particle.

40. A complex between an antibody according to claim 36, an angiogensis activator, and a cytolytic compound.

41. Process for preparing the anti-idiotypic antibodies directed against monoclonal antibodies that are directed against endothelial cells with an angiogenic phenotype, said process comprising the following steps:
immunizing an animal by injection of monoclonal antibodies according to claim 34,
fusing between myelomas of an animal and splenocytes of an animal, in order to obtain hybridomas;
preparing a culture of said hybridomas;
cloning of said hybridomas and secreting antibodies directed against said monoclonal antibodies, said monoclonal antibodies being directed against cells with an angiogenic phenotype, and
verifing the inhibition properties of said antibodies vis-à-vis the function of activation or inhibition of the angiogenesis of the antibodies according to claim 36.

42. Anti-anti-idiotypic antibodies directed against endothelial cells with an angiogenic phenotype according to claim 32, characterized in that said anti-anti-idiotypic antibodies are capable of activating or inhibiting angiogenesis.

43. A process for preparing the anti-anti-idiotypic antibodies of claim 34, directed against endothelial cells with an angiogenic phenotype, said process comprising the following steps:
immunizing an animal by injection of anti-idiotypic antibodies;
fusing between myelomas of an animal and splenocytes of an animal in order to obtain hybridomas;
preparing a culture of the hybridomas thus obtained;
cloning of said hybridomas and secreting anti-anti-idiotypic antibodies directed against cells with an angiogenic phenotype; and
verifing the properties of inhibition or activation of said anti-anti-idiotypic antibodies.

44. A pharmaceutical composition, characterized in that it contains, as active ingredient, an angiogenesis inhibitor, chosen from an antibody according to claim 34, an anti-idiotypic antibody raised against said antibody, a Fab fragment of said antibody or a complex comprising said antibody or said Fab fragment, in combination with a pharmaceutically acceptable vector, said pharmaceutical composition being capable of being administered at a rate of approximately 0.01 to approximately 20 mg/kg/injecction.

45. A vaccine composition comprising as active ingredient a monoclonal antibody according to claim 34, an anti-idiotpyic antibody raised against said antibody, Fab fragments of said antibody, or an anti-anti-idiotypic antibody in combination with a pharmaceutically acceptable adjuvant.

46. A method for the treatment of pathologies requiring inhibition of endothelial proliferation, comprising administering an effective amount of angiogenesis inhibitor to a subject in need thereof, wherein said angiogenesis inhibitor is selected from the group consisting of an antibody according to claim 34, an anti-idiotypic antibody raised against said antibody, a Fab fragment of said antibody, or an anti-anti-idiotypic antibody.

47. A method for the treatment of pathologies requiring the inhibition of endothelial activation, comprising administering an effective amount of angiogenesis inhibitor to a subject in need thereof, wherein said angiogenesis inhibitor is selected from the group consisting of an antibody according to claim 34, an anti-idiotypic antibody raised against said antibody, a Fab fragment of said antibody, or an anti-anti-idiotypic antibody.

48. A method for preparing a medicament intended to promote cicatrization, comprising administering an effective amount of angiogenesis inhibitor to a subject in need thereof, wherein said angiogenesis inhibitor is selected from the group consisting of an antibody according to claim 34, an anti-idiotypic antibody raised against said antibody, a Fab fragment of said antibody, or an anti-anti-idiotypic antibody.
